(19) United States
(12) Patent Application Publication
(10) Pub. No.: US 2012/0247610 A1
(43) Pub. Date: Oct. 4, 2012

(54) METHOD FOR AUTOMATICALLY INSPECTING A BEVERAGE PROCESSING PLANT

(75) Inventor: Kaj Führer, Tegernheim (DE)

(73) Assignee: KRONES AG, Neutraubling (DE)

(21) Appl. No.: 13/438,900

(22) Filed: Apr. 4, 2012

(30) Foreign Application Priority Data
Apr. 4, 2011 (DE) 10 2011 006 717.5

(51) Int. Cl. B67C 3/22; G01N 33/20

(52) U.S. Cl. 141/1; 141/94; 73/61.43

(57) ABSTRACT

A method for automatically inspecting a beverage processing plant including determining at least one measured quantity of a liquid medium by at least one sensor, where the liquid medium is produced, used and/or treated in at least one treatment device of the beverage processing plant, and where the determination is carried out after the production, use and/or treatment of the liquid medium, comparing the determined measured quantity with a predetermined set value for the measured quantity, and, if a deviation of the determined measured quantity from the predetermined set value is detected, determining a cause for the deviation and/or a source of the deviation on the basis of the value of the deviation and/or on the basis of the determined measured quantity.

### CROSS-REFERENCE TO RELATED APPLICATION

The present application claims the benefit of priority of German Application No. 102011006717.5, filed Apr. 4, 2011. The entire text of the priority application is incorporated herein by reference in its entirety.

### FIELD OF THE DISCLOSURE

The present disclosure relates to a method for automatically inspecting a beverage processing plant.

### BACKGROUND

During the operation of beverage processing plants damages to components or contaminations may occur, which have a negative effect on the quality of the produced beverage product. As a rule, this is prevented by providing the individual treatment devices of a beverage processing plant with sensors, allowing to check whether the treatment devices operate properly.

Despite these sensors, it is often impossible to promptly identify the cause for and/or the source of process errors and/or damaged components.

### SUMMARY OF THE DISCLOSURE

Therefore, one aspect of the present disclosure to provide an improved method for automatically inspecting a beverage processing plant.

The disclosure provides for a method for automatically inspecting a beverage processing plant including the steps of:

determining at least one measured quantity of a liquid medium by at least one sensor, wherein the liquid medium is produced, used and/or treated in at least one treatment device of the beverage processing plant, and wherein the determination is carried out after the production, use and/or treatment of the liquid medium,

comparing the determined measured quantity with a predetermined set value for the measured quantity, and,

if a deviation of the determined measured quantity from the predetermined set value is detected,

determining a cause for the deviation and/or a source of the deviation on the basis of the value of the deviation and/or on the basis of the determined measured quantity.

The method permits a fast identification and/or localization of process errors and damaged components on the basis of a comparison of the determined measured quantity with a predetermined set value for the measured quantity, and/or on the basis of the value of the determined measured quantity itself. By this, an improved method for the automatic inspection of a beverage processing plant can be obtained.

Specifically, the method allows to check whether the beverage processing plant operates properly and correctly.

Specifically, the determined cause for the deviation may be one possible cause. Specifically, the determined source of the deviation may be one possible source. In other words, determining the cause and/or the source may be associated with uncertainty.

The deviation may be a desired or an undesired parameter alteration.

A beverage processing plant may be a plant in which one or more beverage products are processed, specifically produced and/or bottled. The beverage product can be, for example, an alcoholic or nonalcoholic beverage, e.g. beer, water, juice or a similar beverage.

The treatment device can be, for example, a mixing device in which two or more starting products are mixed to one beverage product. The at least one treatment device may also comprise a filing device for filling a beverage product into containers, e.g. bottles.

The at least one treatment device may also be a device in which the liquid medium is being used as process medium or use medium. For example, the at least one treatment device may comprise a preform production device, specifically an injection molding device, a temperature control device, specifically a heating device, a blow molding machine for producing plastic containers from preforms, a sterilizing device for sterilizing containers, and/or a rinser.

The at least one treatment device may also comprise a transport device for transporting the liquid medium. The transport device can comprise, for example, at least one pump and/or at least one conduit.

Specifically, the liquid medium may be water, specifically process water, use water and/or waste water of the beverage processing plant. Alternatively, the liquid medium may also be a beverage product being processed in the beverage processing plant, or a starting product of a beverage product being processed in the beverage processing plant.

A measured quantity may be a parameter or a characteristic of the liquid medium which can be determined by at least one sensor. That is, determining the at least one measured quantity may correspond to measuring or determining a value of the at least one parameter or the at least one characteristic.

The predetermined or desired set value may be stored in a storage element of the beverage processing plant. Specifically, the comparison of the determined measured quantity with the predetermined set value can be carried out by a control element of the beverage processing plant. The control element can be, for example, a programmable logic controller or a computer.

Specifically, the cause and/or the source may be determined if the deviation and/or the determined measured quantity meet(s) a predetermined criterion. Thus, for example, small fluctuations of the measured quantity and/or measuring uncertainties may be accommodated.

Specifically, the cause and/or the source can be determined only if the deviation and/or the determined measured quantity meet(s) a predetermined criterion.

The predetermined criterion can define, for example, a tolerance for the deviation, specifically wherein, if the deviation of the determined measured quantity from the predetermined set value is smaller than the tolerance, the predetermined criterion is not met. Only if the deviation is greater than the tolerance can the predetermined criterion be deemed to have been met and can a determination of a cause for the deviation and/or a source of the deviation be carried out.

Also, the predetermined criterion can specify at least one threshold for the measured quantity. Thus, for example, limit values may be included.

Specifically, the predetermined criterion can be met if the determined measured quantity exceeds or falls below the threshold. For example, the predetermined set value can correspond to an ideal or desired value for the measured quantity, and the threshold may correspond to a limit value. In this case, the determination of a cause and/or a source can be carried out, on the one hand, if the determined measured quantity deviates from the predetermined set value and, at the same time, exceeds or falls below the threshold.

The predetermined set value may also correspond to the threshold.

Specifically, the determination of a cause for the deviation and/or a source can be carried out by a control element of the beverage processing plant.

A determined cause and/or a determined source may be outputted to an operator, for example, by an indicating device. In the event of a deviation, also a warning message and/or an alarm signal may be outputted, specifically if the deviation and/or the determined measured quantity meet(s) a predetermined criterion, e.g. a predetermined criterion as described above. Specifically, the indicating device may comprise a display screen.

Based on the outputted determined cause and/or the outputted determined source the operator can then initiate further steps, for example, change or adapt at least one operational setting of the beverage processing plant.

Specifically, the determination of a source may comprise a determination or identification of a component of the beverage processing plant, specifically of a treatment device of the beverage processing plant. Specifically, each component may be assigned a unique measured quantity/characteristic value, so that errors at the end of a process chain are uniquely assignable by a measured deviation, or can at least be locally limited by narrowing down the assignment. The assignment is narrowed down, for example, if unforeseen temperature deviations can be assigned to multiple heating elements. Hence, an operator can at least know where to look for malfunctions.

The determination of a cause for the deviation may comprise specifying a malfunction or misadjustment of one or more components of the beverage processing plant.

Specifically, a cause for the deviation and/or a source may be determined on the basis of the type of the determined measured quantity and/or the magnitude of the deviation of the determined measured quantity from the predetermined set value.

Specifically, the at least one measured quantity can be measured inline. In other words, the measurement of the at least one measured quantity can be carried out in the beverage processing plant, specifically without stopping the beverage processing plant. Thus, possible error sources can be identified faster, permitting a minimization of product losses.

Specifically, the beverage processing plant may comprise multiple treatment devices, wherein the determination of the at least one measured quantity is carried downstream of multiple treatment devices, specifically downstream of all treatment devices.

The liquid medium may be process water, use water and/or waste water. Specifically, the determination of the at least one measured quantity is carried prior to, specifically immediately prior to and/or during the introduction of the process water, use water and/or waste water into a collection system and/or waste water system.

The collection system and/or waste water system may form a part of the beverage processing plant. Alternatively or additionally, the collection system and/or waste water system may also form a part of a waste water system, specifically of a public one.

The liquid medium may be a beverage product produced and/or treated in the beverage processing plant, specifically wherein the determination of the at least one measured quantity is carried out prior to, specifically immediately prior to and/or during the filling of the beverage product into a collection tank and/or into containers, specifically bottles. Hence, in this case, too, the determination of the at least one measured quantity can only be carried out behind or downstream of possible deviation sources.

The at least one measured quantity may comprise a temperature, a concentration, a pressure, a pressure drop and/or a pH-value. For example, the measured quantity can comprise a concentration of a chemical substance, a concentration of residues of seals, a concentration of one or more harmful substances, a pipe pressure, a pressure drop in the liquid medium and/or a concentration of one or more ingredients of a beverage product. The at least one measured quantity may also comprise an optical property of the liquid, e.g. color and/or turbidity.

The determination of a cause and/or source can be carried out on the basis of properties and/or operational settings of the beverage processing plant.

Specifically, the determination of a possible cause and/or a possible source can be carried out on the basis of expert knowledge about the beverage processing plant. In this context expert knowledge may be a specification of properties and/or operational settings of the beverage processing plant, and a specification of correlations of possible values of measured quantities with the properties and/or operational settings. Specifically, the expert knowledge may be implemented or stored in a control device of the beverage processing plant.

In other words, a cause for and/or a source of the deviation can be determined on the basis of the knowledge about the structure of the beverage processing plant and/or the processing procedure in which the liquid medium is produced, used and/or treated.

Furthermore, if a deviation is detected, at least one operational setting can be altered or adapted on the basis of the value of the deviation and/or on the basis of the determined measured quantity. Thus, an automatic reaction to the deviation from the set value is possible.

For example, it is possible to alter or adapt a process parameter for producing, using and/or processing the liquid medium. For example, a method for neutralizing the liquid medium may be altered and/or adapted. Specifically, the liquid medium may be diverted, for example, into another collection tank.

Also, it is possible to stop the beverage processing plant.

In addition, the disclosure provides for a beverage processing plant, including:

at least one treatment device in which a liquid medium is produced, used and/or treated,

at least one sensor for determining at least one measured quantity of the liquid medium, wherein the at least one sensor is disposed downstream of the at least one treatment device, and

a control device adopted and/or configured

to compare the determined measured quantity with a predetermined set value for the measured quantity, and,

if it detects a deviation of the determined measured quantity from the predetermined set value,

determines a cause for the deviation and/or a source of the deviation on the basis of the value of the deviation and/or on the basis of the determined measured quantity.

A beverage processing plant of this type allows a fast identification and/or localization, for example, of process errors and/or damaged components.

In other words, the beverage processing plant, specifically the control device, may be configured to allow the realization of or realize an above-described method for an automatic inspection. In other words, the beverage processing plant is capable of implementing an above-described method.

Specifically, the beverage processing plant, the at least one treatment device, the liquid medium, the at least one measured quantity and/or the predetermined set value may comprise one or more of the aforementioned features. Specifically, also an above-described predetermined criterion may be implemented.

Specifically, the liquid medium may be process water, use water and/or waste water, wherein the beverage processing plant further comprises a device for introducing process water, use water and/or waste water into a collection system and/or waste water system, and wherein the at least one sensor is disposed upstream of, specifically immediately upstream of and/or in the device for introducing process water, use water and/or waste water into a collection system and/or waste water system. The collection system and/or waste water system may form a part of the beverage processing plant or part of a waste water system, specifically a public one.

The liquid medium may also be a beverage product produced and/or treated in the beverage processing plant, wherein the beverage processing plant further comprises a filling device for filling the beverage product into a collection tank and/or into containers, specifically bottles, and wherein the at least one sensor is disposed upstream of, specifically immediately upstream of and/or in the filling device.

### BRIEF DESCRIPTION OF THE DRAWINGS

Other advantages and features of the disclosure will be explained by means of the exemplary figures below, wherein:

FIG. 1 shows an exemplary beverage processing plant; and

FIG. 2 shows another exemplary beverage processing plant.

### DETAILED DESCRIPTION OF THE PREFERRED EMBODIMENT

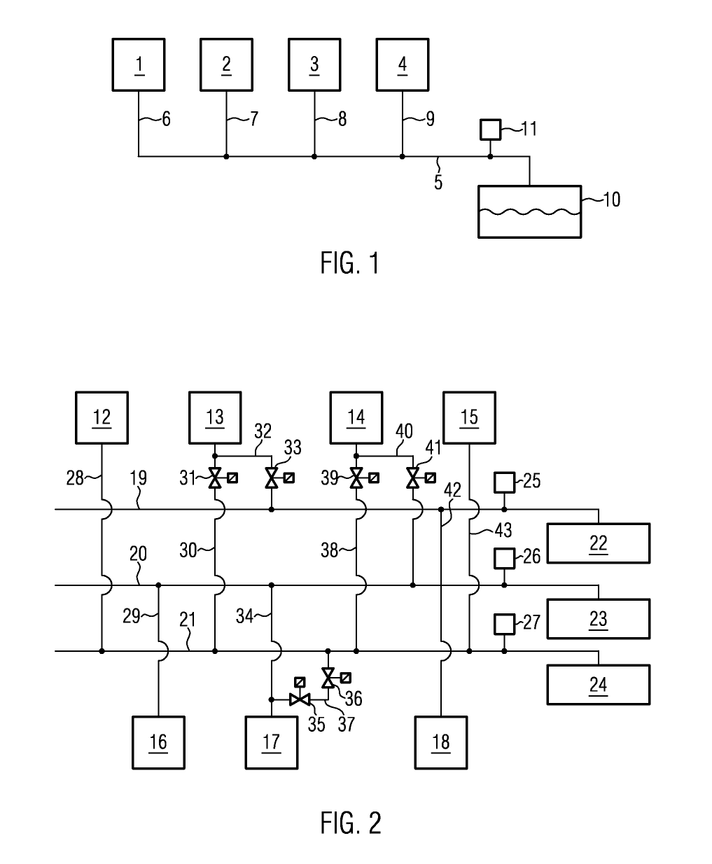

FIG. 1 illustrates a portion of an exemplary beverage processing plant, which can be, for example, a CIP (Cleaning In Place) device of a beverage processing plant, where four collection tanks 1, 2, 3 and 4 for cleaning liquids are provided. The respective liquids can be introduced through feed lines 6, 7, 8 and 9 into one common conduit 5. The different liquids may have different temperatures. The mixture of the cleaning liquids may be conducted through the common conduit 5 into a collection tank 10.

The collection tank 10 can be, for example, a waste water tank. In the present case, the cleaning liquids were conducted through one or more treatment devices (not shown in FIG. 1) of the beverage processing plant in order to be cleaned, before introducing them into the collection tank 10.

Alternatively, the collection tank 10 may collect a cleaning liquid mixed from the four liquids and provide it to non-illustrated downstream treatment devices of the beverage processing plant.

FIG. 1 further shows a sensor 11 which serves to measure at least one measured quantity of the liquid present in conduit 5. In the present case, the sensor 11 is disposed immediately upstream of the collection tank 10 and downstream of possible treatment devices of the beverage processing plant. The sensor 11 can be, for example, a temperature sensor. The sensor 11 could also be an optical sensor, however, for determining an optical property of the liquid e.g. the color and/or the turbidity. Alternatively or additionally, the pH-value of the liquid or the concentration of a component of the liquid, e.g. a harmful substance, could be determined.

The measured quantity determined by sensor 11 can be compared to a stored, predetermined set value in a non-illustrated control device of the beverage processing plant.

If the control device detects a deviation of the determined measured quantity from the predetermined set value according to a predetermined criterion, the control device is capable of determining a cause for the deviation and/or a source of the deviation on the basis of the value of the deviation and/or on the basis of the measured quantity.

In other words, it can determine a cause and/or a source if the deviation meets a predetermined criterion. The predetermined criterion can specify, for example, a threshold for the determined measured quantity and/or a tolerance for the deviation.

Specifically, the determination of the cause and/or the source can be determined on the basis of the knowledge of the processes, specifically the process time and/or the travel of the liquid medium through the conduits. That is, the determination can be carried out by means of assigning the medium flows/medium courses and/or cycle times/process times to sources, processes, machines and/or components where the liquid medium is treated and/or modified in any way. Alternatively or additionally, the type of the measured quantity and/or the magnitude of the deviation allow(s) a conclusion to the source of and/or the cause for the deviation.

Such a method allows a fast identification and/or localization, for example, of process errors and damaged components. Thus, a reduction of repair times can be achieved, e.g. mean times to repair (MTTR).

Also, according to the predetermined criterion, an alarm signal may be outputted to an operator even if the deviation is only a small one, for example, if the measured value exceeds or falls below a predetermined threshold.

In the case of the example illustrated in FIG. 1, for example, a measured temperature of the medium in conduit 5 can be compared automatically with a predetermined set value. If a deviation is detected it can be concluded automatically, e.g. by means of the magnitude of the deviation, in which one of the tanks 1 to 4 the cleaning liquid has a wrong temperature. To this end, the knowledge about the mixing ratios and/or the heating processes in the tanks may be used.

FIG. 2 shows another exemplary portion of a beverage processing plant. In this case, multiple common conduits 19, 20 and 21 are illustrated, each leading into one collection tank 22, 23 and 24, respectively. Treatment devices 12 to 18 may conduct liquid media into one or more of the collection conduits 19, 20, and 21. For example, a liquid may be conducted from treatment device 12 through the feed line 28 into the collection conduit 21. Correspondingly, a feed line 29 is provided for treatment device 16.

Treatment devices 13, 14 and 17 each have two feed lines. For example, a liquid may be introduced from the treatment device 13 through the feed line 30 into the common conduit 21. Alternatively, the liquid may be conducted through the feed line 32 into the common conduit 19. The feed line into one or another of the common conduits can be controlled by means of valves 31 and 32. Similarly, the treatment device 14 comprises two feed lines 38 and 40 with valves 39 and 41. The treatment device 17 comprises feed lines 34 and 37. In this case, two valves 35 and 36 are provided in the second feed line 37 leading into the common conduit 21.

Sensors 25, 26 and 27 are each provided immediately upstream of the collection tanks 22 to 24, by means of which at least one measured quantity of the liquid medium present in the collection conduit 19, 20 or 21 can be determined. A non-illustrated control device can then carry out an above-described method for checking the function of the beverage processing plant on the basis of the determined measured quantities, wherein the three liquid media can be regarded either individually or in combination.

It will be appreciated that features recited in the above-described exemplary embodiments are not limited to these specific combinations, and are realizable in any other combinations, too.

What is claimed is:

1. A method for automatically inspecting a beverage processing plant, comprising:
determining at least one measured quantity of a liquid medium by at least one sensor, wherein the liquid medium is one of produced, used, treated, and a combination thereof in at least one treatment device of the beverage processing plant, and wherein the determination is carried out after the production, use, treatment, and a combination thereof of the liquid medium;
comparing the determined measured quantity with a predetermined set value for the measured quantity; and,
if a deviation of the determined measured quantity from the predetermined set value is detected,
determining one of a cause for the deviation, a source of the deviation, and combination thereof one of on the basis of the value of the deviation, on the basis of the determined measured quantity, and on the basis of a combination thereof.

2. The method according to claim 1, wherein the determination of one of the cause, the deviation or a combination thereof is carried out if the deviation, the determined measured quantity, and a combination thereof meets a predetermined criterion.

3. The method according to claim 1, wherein the liquid medium is one of process water, use water, waste water, and a combination thereof.

4. The method according to claim 1, wherein the liquid medium is a beverage product one of produced, treated, and a combination thereof in the beverage processing plant.

5. The method according to claim 1, wherein the at least one measured quantity comprises a temperature, a concentration, a pressure, a pressure drop, a pH-value, and a combination thereof.

6. The method according to claim 1, wherein the determination of one of a cause, a source, and a combination thereof is carried out on the basis of one of properties, operational settings, and a combination thereof of the beverage processing plant.

7. The method according to claim 6, wherein the determination of one of a cause, a source, and a combination thereof is carried out on the basis of expert knowledge about the beverage processing plant.

8. A beverage processing plant, comprising:
at least one treatment device in which a liquid medium is one of produced, used, treated, and a combination thereof;
at least one sensor for determining at least one measured quantity of the liquid medium, wherein the at least one sensor is disposed downstream of the at least one treatment device; and
a control device adopted and/or configured to compare the determined measured quantity with a predetermined set value for the measured quantity; and,
if the control device detects a deviation of the determined measured quantity from the predetermined set value,
to determine one of a cause for the deviation, a source of the deviation, and a combination thereof one of on the basis of the value of the deviation, on the basis of the determined measured quantity, and on the basis of a combination thereof.

9. The beverage processing plant according to claim 8, wherein the liquid medium is one of process water, use water, waste water, and a combination thereof, wherein the beverage processing plant further comprises a device for introducing one of the process water, water, waste water, and a combination thereof into one of a collection system, a waste water system, and a combination thereof and wherein the at least one sensor is disposed one of upstream of, in the, and a combination thereof, the device for introducing the one of process water, use water, waste water, and a combination thereof into one of the collection system, waste water system, and a combination thereof.

10. The beverage processing plant according to claim 8, wherein the liquid medium is a beverage product one of produced, treated, and a combination thereof in the beverage processing plant, and further comprising a filling device for filling the beverage product into one of a collection tank, containers, and a combination thereof, and wherein the at least one sensor is disposed upstream of the filling device, in the filling device, and a combination thereof.

11. The method according to claim 3, wherein the determination of the at least one measured quantity is carried out one of prior to, during, and a combination thereof the introduction of one of the process water, use water, waste water, and a combination thereof into one of a collection system, a waste water system, and a combination thereof.

12. The method according to claim 11, and wherein the determination of the at least one measured quantity is carried out immediately prior to the introduction of one of the process water, use water, waste water, and a combination thereof.

13. The method according to claim 4, and wherein the determination of the at least one measured quantity is carried out one of prior to, during, and a combination thereof the filling of the beverage product into one of a collection tank, the containers, and a combination thereof.

14. The method according to claim 13, and wherein the determination of the at least one measured quantity is carried out immediately prior to the filling of the beverage product.

15. The method according to claim 13, wherein the containers are bottles.

16. The beverage processing plant according to claim 9, wherein the at least one sensor is disposed immediately upstream of the device for introducing.

17. The beverage processing plant according to claim 10, wherein the containers are bottles.

18. The beverage processing plant according to claim 10, wherein the at least one sensor is disposed immediately upstream of the filling device.
